SEQUENCE LISTING (1 sequence)

SEQ ID NO: 1 - protein, 160 aa, synthetic construct
MHHHHHHQVQLQESGGGLVQPGGSLRLSCAASGSSISINNWGWYRQAPGKQRERVAAISGGGKTVYADSVKGRFTISRDNAKNTVYLQMNSLKPEDTAVYYCRAVRSTGWLRGLDVWGQGTQVTVSAEPKTPKPQPAAAHHHHHHHHYTDIEMNRLGKGA

(19) United States
(12) Patent Application Publication
(10) Pub. No.: US 2026/0108636 A1
(43) Pub. Date: Apr. 23, 2026

(54) MULTI-MODAL MOLECULAR IMAGING NANOPROBE FOR EARLY WARNING AND DYNAMIC MONITORING OF ATHEROSCLEROTIC PLAQUES AND USE THEREOF

(71) Applicant: THE SIXTH MEDICAL CENTER OF CHINESE PLA GENERAL HOSPITAL, Beijing (CN)

(72) Inventors: Yundai CHEN, Beijing (CN); Mingrui MA, Beijing (CN); Yingqian ZHANG, Beijing (CN); Suhui ZHANG, Beijing (CN); Wei TONG, Beijing (CN); Rundou CHEN, Beijing (CN); Hao ZHOU, Beijing (CN); Zhongxuan LI, Beijing (CN); Ziqian WANG, Beijing (CN)

(73) Assignee: THE SIXTH MEDICAL CENTER OF CHINESE PLA GENERAL HOSPITAL, Beijing (CN)

(21) Appl. No.: 19/336,412

(22) Filed: Sep. 22, 2025

Related U.S. Application Data
(63) Continuation of application No. PCT/CN2024/077142, filed on Feb. 9, 2024.

(30) Foreign Application Priority Data
Apr. 24, 2023 (CN) 202310445747.7

(51) Int. Cl. A61K 49/18; A61K 49/00; A61K 49/16; A61P 9/10

(52) U.S. Cl. CPC A61K 49/1866; A61K 49/0032; A61K 49/0058; A61K 49/16; A61P 9/10

(57) ABSTRACT

The present invention belongs to the technical field of fluorescence detection, and specifically relates to a multi-modal molecular imaging nanoprobe for early warning and dynamic monitoring of atherosclerotic plaques and the use thereof. The multi-modal molecular imaging nanoprobe for early warning and dynamic monitoring of atherosclerotic plaques is Fe₃O₄-A12-Cy7. The probe of the present invention is Fe₃O₄-A12-Cy7, wherein protein A12 is a single-domain antibody that can specifically bind to PlexinD1, that is, probe Fe₃O₄-A12-Cy7 can specifically bind to PlexinD1, thus achieving the optimization of the target. According to the present invention, FLI/MPI/CTA is fused to form multi-modal imaging. The multi-modal imaging has the advantages of a high sensitivity and a high spatial resolution, can realize early warning and dynamic monitoring of atherosclerotic plaques, and can also visually reflect 3D stereoscopic imaging, thereby providing theoretical support and technical support for basic research.

### CROSS-REFERENCE TO RELATED APPLICATIONS

The application claims priority to Chinese patent application No. 202310445747.7, filed on Apr. 24, 2023, the entire contents of which are incorporated herein by reference.

### SEQUENCE LISTING

The sequence listing xml file submitted herewith, named “SeqList.xml”, created on Dec. 25, 2025, and having a file size of 2,380 bytes, is incorporated by reference herein.

### TECHNICAL FIELD

The present invention belongs to the technical field of fluorescence detection, and specifically relates to a multi-modal molecular imaging nanoprobe for early warning and dynamic monitoring of atherosclerotic plaques and the use thereof.

### BACKGROUND

The “Annual Report on Cardiovascular Health and Diseases in China (2021)” shows that the incidence and mortality rate of cardiovascular disease in China still ranks first. There are about 330 million people suffering from cardiovascular diseases, including 13 million of cerebral apoplexy (also known as stroke) and 11.39 million of coronary heart diseases. In 2019, the causes of death from cardiovascular diseases in rural areas and cities accounted for 46.74% and 44.26% of the total mortality respectively, that is, on average, two out of every five deaths were caused by cardiovascular diseases. Therefore, the situation of cardiovascular disease prevention and control is still very serious.

Atherosclerotic plaque is the main pathological change in cardio- and cerebrovascular events such as ischemic heart disease and stroke. Atherosclerosis (AS) is a chronic inflammatory lesion affected by multiple factors. The cellular components and overall structure thereof in plaques are an important basis for accurate plaque staging, early warning of the risk of cardiovascular event, and evaluation of intervention efficacy, and also important information for the study of atherosclerotic plaque progression. However, current equipment for plaque evaluation at the microscopic cellular level is limited to single sample, cross-sectional, and single-component imaging, which cannot meet the needs of basic research on atherosclerotic plaques. Molecular imaging can use special probes to display specific molecules and cells in living bodies, and conduct qualitative and quantitative research on their biological behaviours to achieve continuous and dynamic monitoring of living animals. Molecular imaging can not only be used as a practical and convenient means of basic medical research, but also can provide effective evidence for the occurrence, development, prognosis and early diagnosis of clinical diseases, acting as a bridge between molecular biology and clinical medicine.

Currently, clinical imaging methods for plaque identification include CTA, ultrasound, MRI, angioscopy, OCT, IVIS, PET, SPECT, etc. Each of these traditional imaging methods has its own advantages and disadvantages. For example, although CTA is non-invasive, it involves radiation exposure, and histological complexity will reduce its detection accuracy. MRI is non-invasive and non-radiative, but has limited spatial resolution and low sensitivity. It is easily affected by many factors such as respiration and heartbeat, incomplete blood flow transplantation and incomplete fat saturation, and requires cross-sectional contrast to measure stenosis. Angioscopy, OCT, and IVIS can observe the components and properties of plaques, but they are all invasive and expensive, making it difficult to continuously and dynamically monitor the occurrence and development of plaques. PET is unable to provide information about anatomical structures and has low resolution. Multi-modal imaging that fuses two or more imaging methods can achieve complementary advantages. For example, the radioactive tracer used in SPECT/PET imaging has very high sensitivity, and combined application with the radioactive tracer can make up for the lack of imaging sensitivity. Current multimodal imaging that combines two or more imaging modalities can greatly improve the early diagnosis of vulnerable plaques in AS.

The invention patent with the publication number CN110302400B discloses a 64Cu-NOTA-Fe₃O₄-MMPsC-PEG multi-modal nano-intelligent probe constructed by coupling MMP-cleavable polypeptide (Ac-GPLGVRGKC-N12, MMPsC) and 64Cu on the surface of IONP particles, wherein MMPsC can detect the MMPs enzyme activity in atherosclerotic plaques through the interaction between enzyme and substrate. As a magnetic resonance enhancer, iron oxide nanoparticles have small particle size and strong penetrating ability. They can significantly shorten the T2 relaxation time of MRI and thus improve the signal-to-noise ratio; 64Cu enables PET/CT imaging. However, this method has the following limitations: (1) the target molecules are matrix metalloproteinases (MMPs enzymes), which cannot directly reflect the M1 macrophage components of plaques, and are not suitable for basic research focusing on other molecular mechanisms: (2) the imaging method is PET/MRI/CT, which has limited sensitivity and spatial resolution, and the operation is complicated. PET/CT acquisition takes a long time, and MRI image processing requires layer-by-layer comparison to calculate CNR changes %, which fails to achieve more visual 3D stereoscopic imaging. (3) the atherosclerotic mice used for the imaging are induced by high-fat diet feeding for one month and then continuing to be fed with high-fat diet for three months after carotid artery ligation, but earlier warning and continuous dynamic monitoring of atherosclerotic plaques failed to materialize.

Therefore, there is a need to develop a FLI/MPI/CTA multi-modal molecular imaging nanoprobe that enables early warning and dynamic monitoring of atherosclerotic plaques.

### SUMMARY

One of the purposes of the present invention is to provide a multi-modal molecular imaging nanoprobe for early warning and dynamic monitoring of atherosclerotic plaques, so as to solve the problem that the prior art fails to achieve earlier warning and continuous dynamic monitoring of atherosclerotic plaques.

The technical solution of the present invention for a multi-modal molecular imaging nanoprobe for early warning and dynamic monitoring of atherosclerotic plaques is as follows:
a multi-modal molecular imaging nanoprobe for early warning and dynamic monitoring of atherosclerotic plaques, and the nanoprobe is Fe₃O₄-A12-Cy7.
preferably, the A12 in the nanoprobe Fe₃O₄-A12-Cy7 is a A12 protein, and the sequence of the A12 protein is shown in SEQ ID NO:1; SEQ ID NO:1 is as follows: MHHHHHHQVQ LQESGGGLVQ PGGSLRLSCA ASGSSISINN WGWYRQAPGK QRERVAAISG GGKTVYADSV KGRFTISRDN AKNTVYLQMN SLKPEDTAVY YCRAVRSTG WLRGLDVWGQ GTQVTVSAEP KTPKPQPAAA HHHHHHHHYT DIEMNRLGKG A.

A second purpose of the present invention is to provide a method for preparing a multi-modal molecular imaging nanoprobe for early warning and dynamic monitoring of atherosclerotic plaques. A method for preparing a multi-modal molecular imaging nanoprobes for early warning and dynamic monitoring of atherosclerotic plaques, including the following steps:
(1) pretreatment comprising leaving a Fe₃O₄ solution to stand, then discarding a supernatant, washing with MES and performing resuspension;
(2) preparation of a magnetic nanoparticle Fe₃O₄-A12, comprising adding EDC into the Fe₃O₄ obtained in step (1) under vortexing, uniformly mixing and then adding A12, and uniformly mixing and then conducting a reaction under shaking; after the reaction is completed, taking out a reaction liquid, centrifuging, discarding a supernatant, and washing with purified water to obtain the magnetic nanoparticle Fe₃O₄-A12;
(3) preparation of a nanoprobe Fe₃O₄-A12-Cy7, comprising adding Cy7-NHS to the magnetic nanoparticle Fe₃O₄-A12 obtained in step (2), reacting under shaking, centrifuging, discarding a supernatant, and washing with purified water to obtain the nanoprobe Fe₃O₄-A12-Cy7.
preferably, in step (1), the particle size of Fe₃O₄ in the Fe₃O₄ solution is 5 to 30 nm, and the concentration of the Fe₃O₄ solution is 0.01 to 20 mg/mL.
preferably, in step (2), the method for preparing the A12 includes the following steps: \textcircled{1} inserting an A12 gene into an expression vector pET30a, and then transforming same to a BL21 expression strain; \textcircled{2} spreading the expression strain containing A12 plasmid onto an LB plate which is placed upside down in an incubator; \textcircled{3} selecting a single clone from the plate above, inoculating the single clone into an LB culture medium for culture, adding IPTG, and then inducing expression; \textcircled{4} centrifuging a culture liquid after induction, removing a supernatant, adding a PBS solution to resuspend a precipitate, adding a SDS-PAGE loading buffer, heating a sample, then centrifuging and taking a supernatant for electrophoresis.

Further preferably, in step (2), the mass ratio of the Fe₃O₄ to the A12 is 5 to 20; the temperature of the reaction is 28-40° C., and the reaction time is 10-30 h.

Preferably, in step (3), the mass ratio of the Fe₃O₄ to the Cy7 is 10 to 50:1.

Further preferably, in step (3), the particle size of the nanoprobe Fe₃O₄-A12-Cy7 is 5 to 40 nm; the potential of the nanoprobe Fe₃O₄-A12-Cy7 is −27 to 1 mV.

A third purpose of the present invention is to provide a use of a multi-modal molecular imaging nanoprobe for early warning and dynamic monitoring of atherosclerotic plaques.

Use of a multi-modal molecular imaging nanoprobe for early warning and dynamic monitoring of atherosclerotic plaques in the preparation of an early warning reagent or a diagnostic reagent for atherosclerotic plaques.

Use of a multi-modal molecular imaging nanoprobe for early warning and dynamic monitoring of atherosclerotic plaques in the preparation of a therapeutic efficacy monitoring reagent for atherosclerotic plaques.

### BENEFICIAL EFFECTS

(1) Optimization of targets: PlexinD1 can bind to its classic ligand sema3e, and in terms of the nervous system, it can participate in axon guidance; in terms of the cardiovascular system, it can participate in the cardiovascular development and can also inhibit blood vessel sprouting and angiogenesis by inhibiting the proliferation and migration of endothelial cells; in terms of the immune system, it is related to the migration and adhesion of white blood cells (NK, DC, neutrophils, macrophages), and it promotes the progression of inflammation as a macrophage retention factor. Studies have shown that under the action of the pro-inflammatory factor oxLDL/hypoxia during plaque progression, sema3e is secreted by M1 macrophages and acts on their own PlexinD1 receptors, thereby promoting inflammation and plaque progression by changing the cytoskeleton and reducing the migration out from plaques under the action of CCL2 and CCL19.

In addition to binding to the ligand sema3e, the latest research shows that PlexinD1 can also serve as a mechanosensor on endothelial cells and can sense mechanical effects. Through changes in morphological bending, it binds to NRP1/VEGFR2 to form a complex, promote the binding of VEGFR2 to serine tyrosine kinase, and phosphorylate VEGFR2, thereby causing changes in a series of downstream signals of endothelial cells, thus affecting the specificity of plaque distribution at sites with different shear stress.

Preliminary research results show that PlexinD1 is also closely related to the polarization of macrophages. The specific evidence is as follows: at the animal level, PlexinD1, sema3e, and M1 macrophages are highly colocalized in atherosclerotic plaques in carotid bifurcations; at the cellular level, PlexinD1 is significantly upregulated after M1 macrophages polarization induced by LPS or oxLDL; after PlexinD1 is intervened, macrophage polarization towards M1 subtype will be reduced. The probe of the present invention is Fe₃O₄-A12-Cy7, wherein protein A12 is a single-domain antibody that can specifically bind to PlexinD1, that is, probe Fe₃O₄-A12-Cy7 can specifically bind to PlexinD1, thus achieving the optimization of the target.

(2) Optimization of imaging platform: In vivo fluorescence imaging (FLI) has the advantages of high sensitivity, no ionization, and relatively simple operation. Magnetic particle imaging (MPI), as an emerging tomography method, uses superparamagnetic iron oxide nanoparticles (SPION) as the imaging agent and has high sensitivity (millimolar level), high spatial resolution (submillimetre level), zero tissue depth signal attenuation and no ionizing radiation, and iron oxide nanoparticles are currently approved for clinical applications. Computed tomographic angiography (CTA) can provide anatomical information about blood vessels and directly assess the severity of luminal stenosis. The present invention fuses FLI/MPI/CTA to develop multi-modal imaging. This multi-modal imaging can accurately identify plaques due to its high sensitivity and high spatial resolution, and can be used as a promising imaging method. At the same time, IVIS and MPI images can achieve 3D fused imaging with CT, which is more visual. The multi-modal imaging of the present invention not only has the advantages of a high sensitivity and a high spatial resolution, can realize early warning and dynamic monitoring of atherosclerotic plaques at a bifurcation site, and can also visually reflect 3D stereoscopic imaging, thereby providing theoretical and technical support for basic research.

(3) Optimization of functional verification: The atherosclerotic mouse model was only induced by feeding ApoE^{-/-} mice with high-fat diet, without carotid artery ligation or other treatments. C57 mice fed normally and mice fed with high-fat diet for 10 w and 20 w were imaged separately to verify the probe's role in early identification and dynamic monitoring of carotid plaques in mice.

### BRIEF DESCRIPTION OF DRAWINGS

FIG. 1 shows a flowchart of Example 1 of the present invention;

FIG. 2 shows expression and purification results of A12 protein in Example 1 of the present invention, wherein panel A shows the expression of A12 protein in BL21 (DE3) as analysed by SDS-PAGE, Lane M: SDS-PAGE protein marker, Lane 0: control (without IPTG), Lane 1: induction at 15° C. for 16 h, Lane 2: supernatant after whole bacteria lysis, Lane 3: precipitate after whole bacteria lysis; and panel B shows the purification result of A12 protein supernatant as analysed by SDS-PAGE, Lane M: SDS-PAGE protein marker, Lane 1: supernatant after whole bacteria lysis and centrifugation, Lane 2: effluent after incubation of supernatant with Ni-IDA, Lanes 3-4: elution fraction of 100 mM imidazole, Lane 5: elution fraction of 300 mM imidazole;

FIG. 3 shows the identification result of A12 protein in Example 1 of the present invention, where panel A is the SDS-PAGE identification result; panel B is the Western Blot identification result, Lane 1: BSA (0.00 μg), Lane 2: A12 protein (1.20 μg), M1: SDS-PAGE marker, M2: western blot marker;

FIG. 4 shows the TEM characterization and detection result of the nanoprobe Fe₃O₄-A12-Cy7 (A) and the control probe Fe₃O₄-BSA-Cy7 (B) in Example 1 of the present invention.

FIG. 5 shows the zeta potential characterization and detection results of Fe₃O₄-PEG-COOH, nanoprobe Fe₃O₄-A12-Cy7 and control probe Fe₃O₄-BSA-Cy7 in Example 1 of the present invention.

FIG. 6 shows the absorbance detection results before and after the reaction between Fe₃O₄ solution and A12 antibody in Example 1 of the present invention.

FIG. 7 shows the ultraviolet excitation spectrum detection results of the nanoprobe Fe₃O₄-A12-Cy7 and the control probe Fe₃O₄-BSA-Cy7 in Example 1 of the present invention.

FIG. 8 shows the IVIS (A) and MPI (B) signal intensity detection results of the nanoprobe Fe₃O₄-A12-Cy7 at different concentrations in Example 1 of the present invention.

FIG. 9 shows the cell viability detection results of the nanoprobe Fe₃O₄-A12-Cy7 at different concentrations in Example 1 of the present invention.

FIG. 10 is a graph showing immunofluorescence results (scal bar, 50 m) demonstrating the targeting ability of the nanoprobe Fe₃O₄-A12-Cy7 by in vitro cell experiment in Example 1 of the present invention.

FIG. 11 shows the IVIS images of the metabolic process at the carotid artery (A) and biodistribution in various organs (B, C) of the nanoprobe Fe₃O₄-A12-Cy7 and the control probe Fe₃O₄-BSA-Cy7 in Example 1 of the present invention.

FIG. 12 shows an in vivo (A), ex vivo (B) IVIS imaging and a 3D IVIS-CT fusion image (C) of the nanoprobe Fe₃O₄-A12-Cy7 targeting plexind1 in carotid atherosclerotic plaques in mouse in Example 1 of the present invention.

FIG. 13 shows an in vivo (A), ex vivo (B) MPI images and a 3D MPI-CTA fusion image (C) of the nanoprobe Fe₃O₄-A12-Cy7 targeting plexind1 in carotid atherosclerotic plaques in mouse in Example 1 of the present invention.

FIG. 14 shows the images of the results of HE staining (A) and plexind1 immunohistochemical staining (B) of the histopathological sections from carotid bifurcation sites in C57 control mice and atherosclerotic mice fed with high-fat diet for 10 w and 20 w in Example 1 of the present invention.

FIG. 15 shows the images of Prussian blue staining results of histopathological sections of atherosclerotic plaques at carotid bifurcation sites after injection of non-targeting control probe Fe₃O₄-BSA-Cy7 (left) and targeting nanoprobe Fe₃O₄-A12-Cy7 (right) for 24 h in atherosclerotic mice fed with high-fat diet for 20 w in Example 1 of the present invention.

FIG. 16 shows the TEM characterization and detection result of the nanoprobe Fe₃O₄-A12-Cy7 in Example 2 of the present invention.

FIG. 17 shows the TEM characterization and detection result of the nanoprobe Fe₃O₄-A12-Cy7 in Example 3 of the present invention.

FIG. 18 shows the TEM characterization and detection result of the nanoprobe Fe₃O₄-A12-Cy7 in Example 4 of the present invention.

FIG. 19 shows the TEM characterization and detection result of the nanoprobe Fe₃O₄-A12-Cy7 in Example 5 of the present invention.

### DETAILED DESCRIPTION OF THE EMBODIMENTS

The technical solution of the present invention will be further described below in conjunction with specific examples.

### Example 1

A method for preparing a multi-modal molecular imaging nanoprobes for early warning and dynamic monitoring of atherosclerotic plaques, including the following steps:

### (1) Preparation of A12 Protein

A12 protein sequence is shown in SEQ ID NO:1: SEQ ID NO:1 is as follows:

MHHHHHHQVQ LQESGGGLVQ PGGSLRLSCA ASGSSISINN
WGWYRQAPGK QRERVAAISG GGKTVYADSV KGRFTISRDN
AKNTVYLQMN SLKPEDTAVY YCRAVRSTG WLRGLDVWGQ
GTQVTVSAEP KTPKPQPAAA HHHHHHHHYT DIEMNRLGKG
A

According to the gene sequence of interest, the codon optimization software MaxCodon™ Optimization Program (V13) was used to optimize the amino acid sequence of the A12 protein provided, and overlapping single-stranded oligonucleotides were designed and synthesized, and then the full length was spliced through overlap extension PCR. The A12 gene was inserted into the expression vector pET30a through restriction enzyme sites NdeI and HindIII, and the accuracy of the final expression vector was confirmed by enzyme digestion and sequencing, and then the confirmed expression vector was transformed to the BL21 (DE3) expression strain.

The constructed expression strain containing the A12 plasmid was evenly spread on an LB plate (containing 50 μg/mL kanamycin sulphate) and then the plate was inverted and placed in a 37° C. incubator overnight. A single clone was selected from the transformed plate and inoculated into 4 mL of LB medium (containing 50 μg/mL kanamycin sulphate). When the OD600 was 0.5-0.8, 0.2 mM IPTG was added to the culture liquid, and then the expression was induced at 15° C. The culture was expanded and when OD600=0.8, 0.2 mM IPTG was added and the bacteria were collected after induction at 15° C. for 16 h. The induced culture liquid was centrifuged at 12000 rpm for 5 min, the supernatant was removed, PBS was added to resuspend the precipitate, and finally SDS-PAGE loading buffer was added and the sample was heated at 100° C. for 10 min, and then centrifuged to obtain the supernatant for electrophoresis.

### (2) Preparation of Probe

\textcircled{1} Two groups of 20 nm Fe₃O₄ solutions (12.5 mg, 1 mg/mL) were taken and placed in centrifuge tubes which were centrifuged at 14000 r/min for 30 min, and the supernatant was discarded. MES was used for washing once, and resuspension was performed to 10 mL.

\textcircled{2} 12.5 μL of EDC (100 mg/mL) was added to the above Fe₃O₄ under vortexing, and the obtained mixture was mixed well, and then 1.25 mg of A12 (0.61 mg/mL, 2.049 mL) or 1.25 mg of BSA (10 mg/mL, 125 μL) was added, then MES was added to a total volume of 12.5 mL, and the obtained mixture was mixed well and reacted under shaking at 37° C. for 20 h. After the reaction was completed, the reaction liquid was taken out, centrifuged, and the supernatant was collected (for detecting the A12 coupling rate). The remaining solid was washed three times with purified water and then the volume was set to 5 mL, thus obtaining two groups of magnetic nanoparticles 20 nm Fe₃O₄-BSA and 20 nm Fe₃O₄-A12.

\textcircled{3} 0.5 mg of Cy7-NIS was added to the above two groups of magnetic nanoparticles 20 nm Fe₃O₄-BSA and 20 nm Fe₃O₄-A12, respectively, and the mixture was reacted under shaking on a shaker at 37° C. for 1 h, and then the supernatant was discarded by centrifugation. The magnetic material was washed 3 times with purified water and then the volume was set to 12.5 mL, that is, 20 nm Fe₃O₄-BSA-Cy7 and 20 nm Fe₃O₄-A12-Cy7 were obtained.

### (3) Construction of Atherosclerosis Model

ApoE^{-/-} mice (6-8 w, male) were fed with customized high-fat diet (15% lard, 1.25% cholesterol, 0.2% sodium cholate lard) for 10 w and 20 w, and ultrasound confirmed that the atherosclerotic plaque model was successfully constructed.

The mice were divided into three groups, namely:
control group: C57 mice+Fe₃O₄-A12-Cy7;
experimental group 1: ApoE^{-/-} mice HFD10 w+Fe₃O₄-A12-Cy7;
experimental group 2: ApoE^{-/-} mice HFD20 w+Fe₃O₄-A12-Cy7.

### (4) In Vivo and Ex Vivo Imaging

### \textcircled{1} Image Acquisition Before Probe Injection

The mice in each group were fasted for 24 h, and the hair in the anterior neck area was removed. After the equipment was initialized, the mice were placed in the anaesthesia box, anaesthetized with isoflurane inhalation for one minute, and then placed in the IVIS dark box, with the head connected to the anaesthesia tube for continuous anaesthesia by inhalation. Operation was performed on the computer, the Cy7 fluorophore was selected, and IVIS 2D images were acquired.

1 μL of Fe3O4-A 12-Cy5 nanoparticles with a Fe concentration of 100 μg/mL was used as a benchmark, sealed in a plastic tube and fixed at the top of the MPI animal bed. The mice were intraperitoneally injected with anaesthetic drugs and fixed on the MPI animal bed. The scanning range was set to (−2±2 cm), 2D, isotropic mode was selected, the number of scans was 3, and MPI 2D images were acquired.

\textcircled{2}Image Acquisition after Probe Injection

24 h after the injection of the corresponding probe via tail vein, the equipment was initialized, then the mice were placed in the anaesthesia box, anaesthetized with isoflurane inhalation for 1 min, and then placed in the IVIS dark box, with the head connected to the anaesthesia tube for continuous anaesthesia by inhalation. Operation was performed on the computer, the Cy7 fluorophore was selected, and IVIS 2D and IVIS3D-CT fusion images were acquired in sequence.

1 μL of Fe₃O₄-A12-Cy5 nanoparticles with a Fe concentration of 100 μg/mL was used as a benchmark, sealed in a plastic tube and fixed at the top of the MPI animal bed. The mice were intraperitoneally injected with anaesthetic drugs and fixed on the MPI animal bed. The scanning range was set to (−2±2 cm), 2D, isotropic mode was selected, the number of scans was 3, and MPI 2D images were acquired.

30 min after the contrast agent was injected via the tail vein, the mice were placed on the animal bed of the CT equipment for CTA image acquisition. The relative position between the mouse and the animal bed was kept unchanged, the animal bed was moved to the MPI equipment, the scanning range was set to (−2±2 cm), 3D, isotropic mode was selected, the number of scans was 1, and the MPI 3D image was acquired, which was later fused with the CTA images.

The carotid arteries and aorta of mice were isolated under stereomicroscope, and IVIS and MPI imaging were performed in the same manner as before.

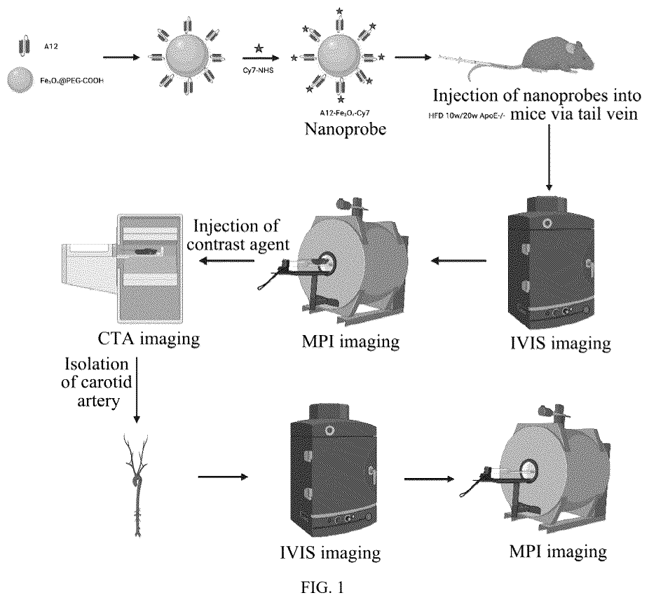

Living Image Software and VivoQuant software were used to analyse and process IVIS and MPI images respectively, where the flow chart is shown in FIG. 1.

### Example 2

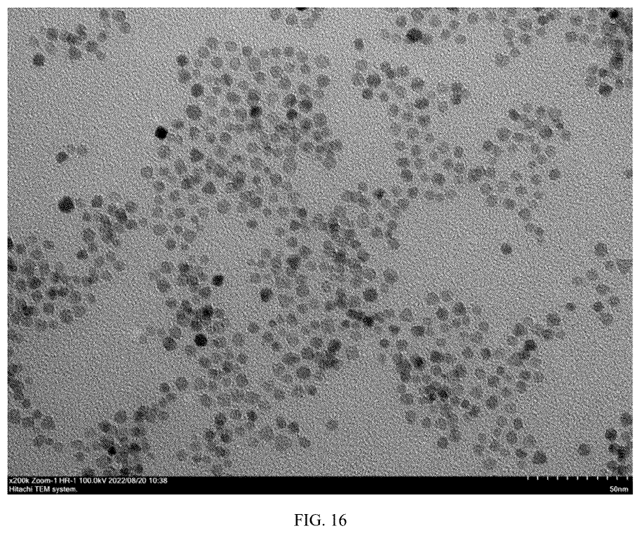

A method for preparing a multi-modal molecular imaging nanoprobe for early warning and dynamic monitoring of atherosclerotic plaques, which was different from Example 1 in that in step (1), the particle size of Fe₃O₄ in the Fe₃O₄ solution was 5 nm, and the concentration of the Fe₃O₄ solution was 20 mg/mL. The obtained nanoprobes were 5 nm Fe₃O₄-BSA-Cy7 and 5 nm Fe₃O₄-A12-Cy7, as shown in FIG. 16.

### Example 3

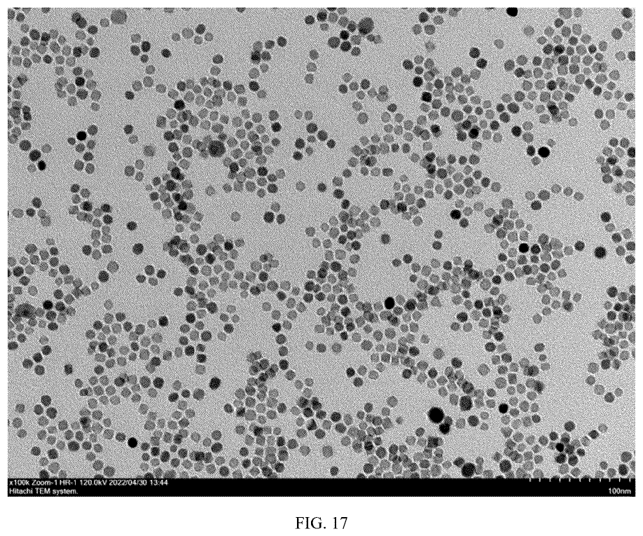

A method for preparing a multi-modal molecular imaging nanoprobe for early warning and dynamic monitoring of atherosclerotic plaques, which was different from Example 1 in that in step (1), the particle size of Fe₃O₄ in the Fe₃O₄ solution was 18 nm, and the concentration of the Fe₃O₄ solution was 9 mg/mL. The obtained nanoprobes were 10 nm Fe₃O₄-BSA-Cy7 and 10 nm Fe₃O₄-A12-Cy7, as shown in FIG. 17.

### Example 4

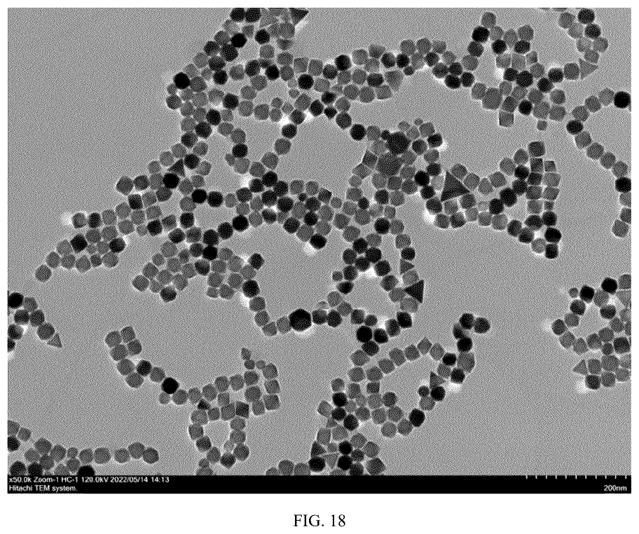

A method for preparing a multi-modal molecular imaging nanoprobe for early warning and dynamic monitoring of atherosclerotic plaques, which was different from Example 1 in that in step (1), the mass of the Fe₃O₄ solution was 25 mg; in step (2), the reaction temperature was 32° C., the reaction time was 26 h, and the obtained nanoprobes were 30 nm Fe₃O₄-BSA-Cy7 and 30 nm Fe₃O₄-A12-Cy7, as shown in FIG. 18.

### Example 5

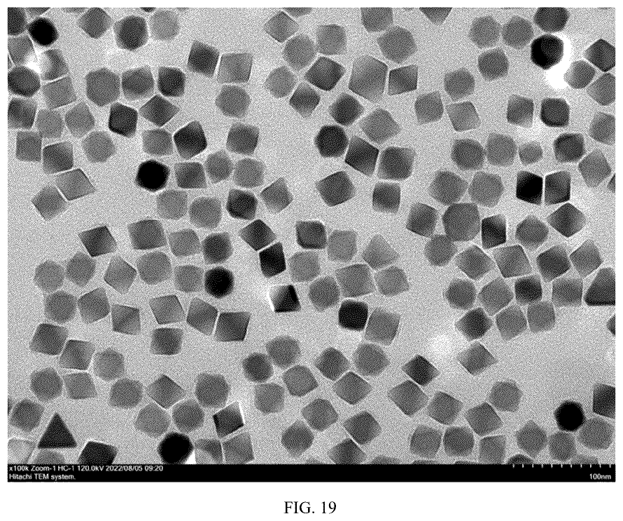

A method for preparing a multi-modal molecular imaging nanoprobe for early warning and dynamic monitoring of atherosclerotic plaques, which was different from Example 1 in that in step (1), the mass of the Fe₃O₄ solution was 6.25 mg; in step (2), the reaction temperature was 28° C., the reaction time was 18 h, and the obtained nanoprobes were 40 nm Fe₃O₄-BSA-Cy7 and 40 mun Fe₃O₄-A12-Cy7, as shown in FIG. 19.

### Results and Analysis

The results in Example 1 were characterized and analysed, and the results are as follows:

### (1) Characterization Results and Analyses of A12 Protein

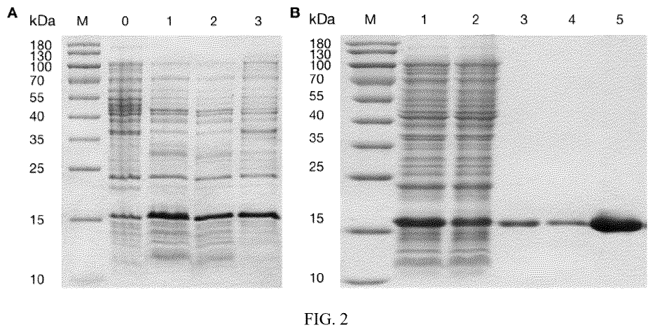

The whole bacteria were lysed by sonication with 20 mM Tris (pH 8.0), 300 mM NaCl, 20 mM Imidazole containing 1% Triton X-100, 1 mM DTT, and 1 mM PMSF. The supernatant and the precipitate were taken for SDS-PAGE analysis and detection. The analysis and detection results are shown in FIG. 2A. The whole bacteria ultrasonic lysis analysis showed that A12 protein was expressed in both the supernatant and inclusion bodies.

The whole bacteria were lysed by sonication with 20 mM Tris (pH 8.0), 300 mM NaCl, 20 mM Imidazole containing 1% Triton X-100, 1 mM DTT, and 1 mM PMSF. The Ni-IDA affinity chromatography column was equilibrated with 20 mM Tris (pH 8.0), 300 mM NaCl, and 20 mM Imidazole buffer. The protein of interest was then eluted with an equilibration buffer containing different concentrations of imidazole, and each elution fraction was collected for SDS-PAGE analysis. The analysis and detection results are shown in FIG. 2B.

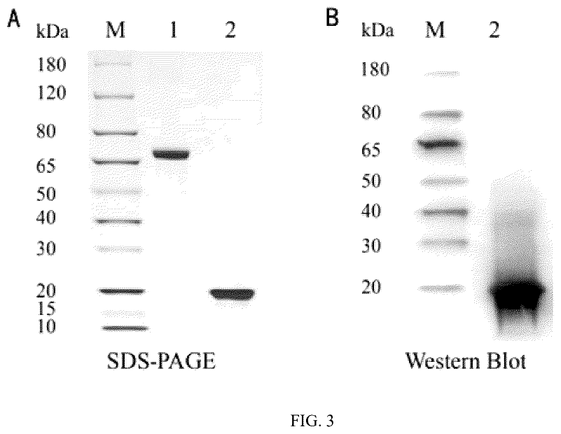

After purification by Ni-IDA affinity chromatography and analysis, Lanes 3-5 with relatively high purity were collected and dialyzed into ultrapure water. After dialysis, it was filtered with a 0.22 μm filter. The SDS-PAGE and WB quality inspection results are shown in FIG. 3. The molecular weight of A12 is about 20 kDa, and the purity is >90%.

### (2) Characterization Results and Analyses of Probe

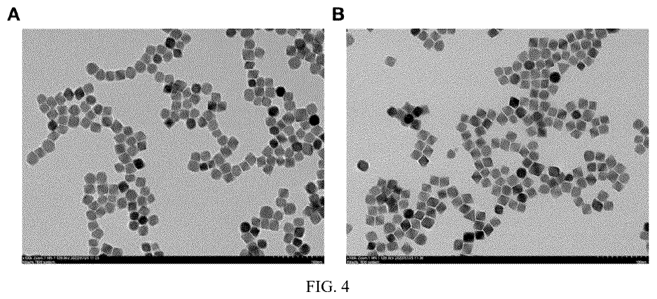

The nanoparticle sizes of Fe₃O₄-BSA-Cy7 and Fe₃O₄-A12-Cy7 were measured using a transmission electron microscope (JEM-1200EX, JEOL, Tokyo, Japan). The results are shown in FIG. 4. As shown in FIG. 4, the nanoparticle sizes of the probes Fe₃O₄-BSA-Cy7 and Fe₃O₄-A12-Cy7 is about 20 nm.

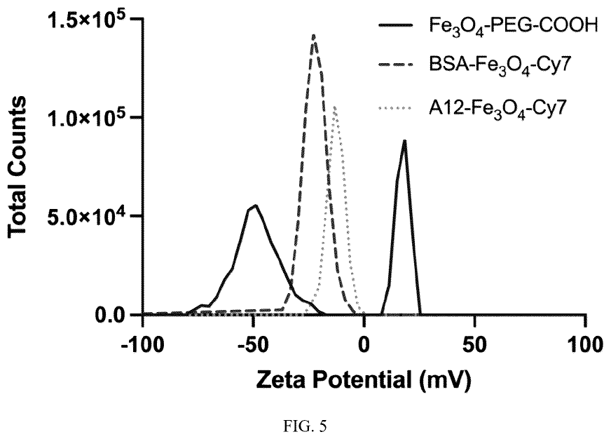

Zetasizer Nano ZS90 (Malvern Instruments, Malvern, UK) was used to measure the dehydrated particle size and potential of the nanoparticles. The results are shown in FIG. 5. It can be seen from FIG. 5 that Fe₃O₄@PEG-COOH is −22.75 mV, Fe₃O₄-BSA-Cy7 is −21.5 mV, and Fe₃O₄-A12-Cy7 is −12.04 mV.

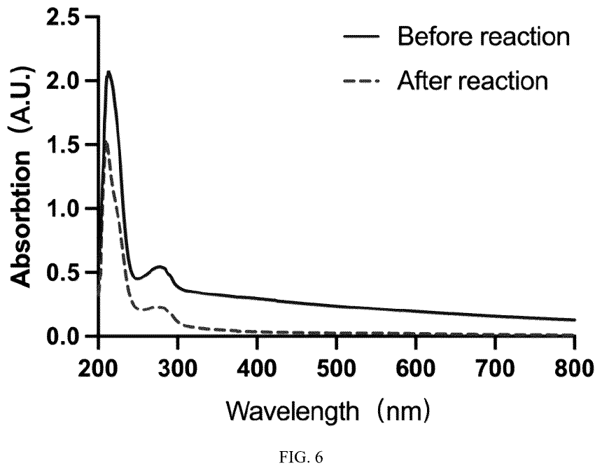

A UV-Vis-NIR spectrophotometre (UV-3600 Plus, Shimadzu, Kumamoto, Japan) was used to measure the optical absorption spectrum of A12 before and after coupling. The results are shown in FIG. 6, and the coupling rate is calculated as 58.4%.

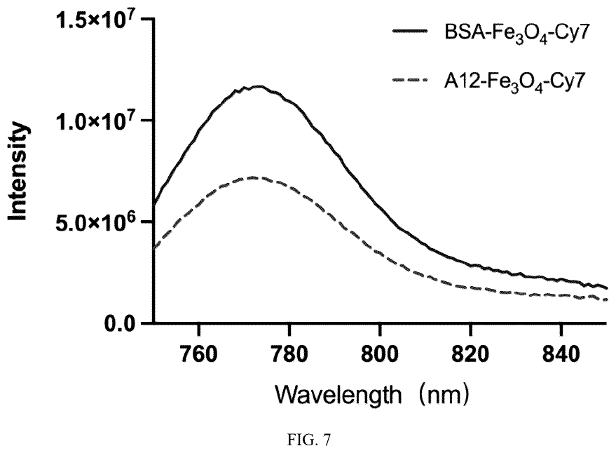

A fluorescence spectrophotometre (F-7000, Hitachi, Tokyo, Japan) was used to measure fluorescence excitation spectra of Fe₃O₄-A12-Cy7 nanoparticles and Fe₃O₄-BSA-Cy7. As shown in FIG. 7, the optimal excitation wavelength of the probe is 760-780 nm.

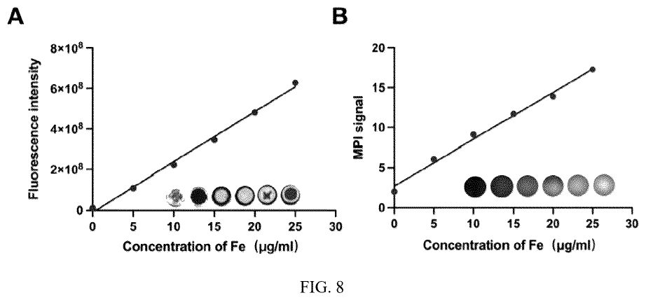

The fluorescence intensity and MPI signals of Fe₃O₄-A12-Cy7 nanoparticles at different concentrations were analysed by IVIS fluorescence imaging equipment (Caliper Life Sciences, PerkinElmer, Waltham, MA, USA) and MPI imager. The results are shown in FIG. 8. As the probe concentration gradient increases, both IVIS and MPI signals increase linearly.

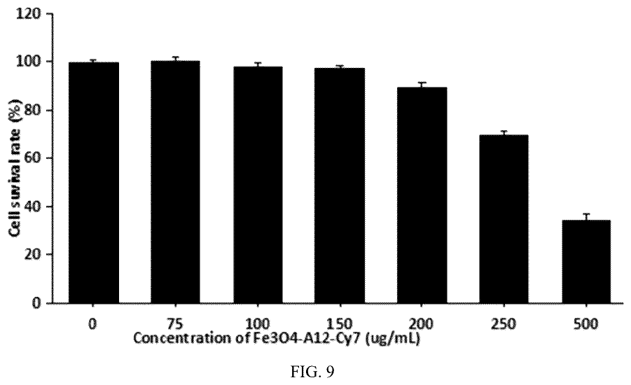

CCK8 was used to measure cytotoxicity. The probe was co-cultured with THP-1 cells at a concentration gradient of 0, 75, 100, 150, 200, 250, and 500 μg/mL for 24 h. The absorbance was measured after adding CCK8, and the cell survival rate was calculated. As shown in FIG. 9, with the increase of material concentration, the cell viability shows a downward trend. When the cells are treated with 200 μg/mL material, the cell viability is still more than 80%.

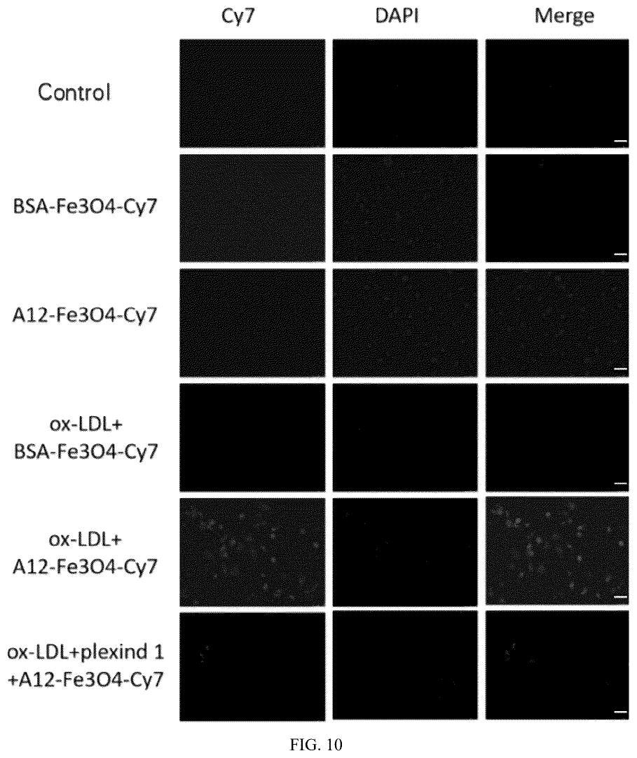

To verify the targeting ability of the probe in cells, THP-1 cells were induced to differentiate and grew on the glass slide, and then divided into six groups: a. macrophages+basal medium; b. macrophages+non-targeting probe Fe₃O₄-BSA-Cy7; c. macrophages+targeting probe Fe₃O₄-A12-Cy7; d. macrophages+oxLDL+non-targeting probe Fe₃O₄-BSA-Cy7; e. macrophages+oxLDL+targeting probe Fe₃O₄-AI2-Cy7; f. macrophages+oxLDL+plexind1 antibody+targeting probe Fe₃O₄-A12-Cy7, the plexind dilution ratio was 1:500, the probe concentration was 50 μg/mL, and the incubation time was 6 h. The slides were fixed, stained with DAPI, and imaged with confocal laser scanning. As shown in FIG. 10, after induction with ox-LDL, the expression of plexind1 was up-regulated, so the fluorescence intensity of the oxLDL+Fe₃O₄-A12-Cy7 group was the strongest; the fluorescence was weakened after treatment with plexind 1 antibody, indicating that the plexind 1 antibody has a competitive relationship with the Fe₃O₄-A12-Cy7 probe.

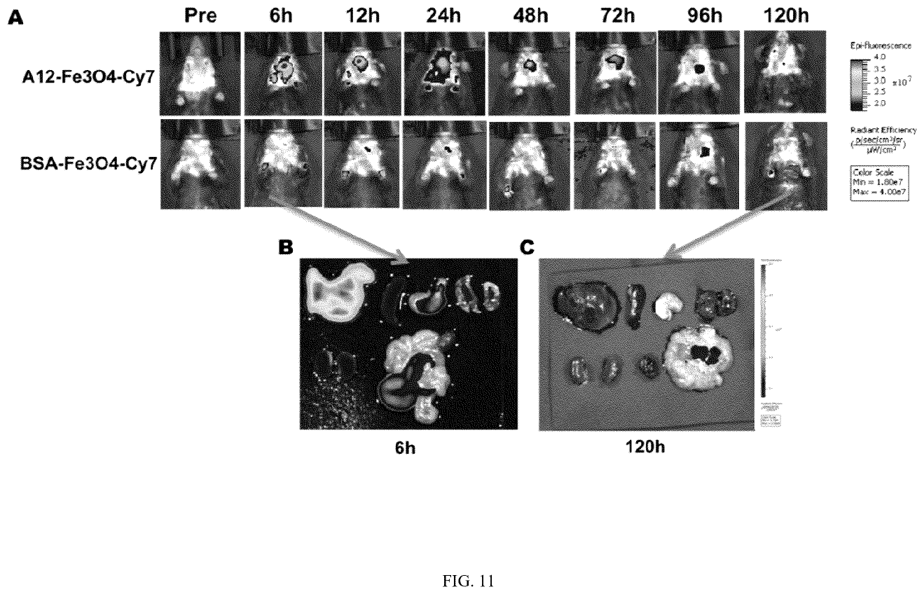

As shown in FIG. 11A, the carotid artery area of the atherosclerotic mouse after injection of the nanoprobe was continuously observed for 120 h. Compared with the non-targeting control probe Fe₃O₄-BSA-Cy7, the signal in the area of interest was significantly increased after injection of the targeting nanoprobe Fe₃O₄-A12-Cy7, and then returned to the pre-injection level at 120 h. The targeting ability of the nanoprobe Fe₃O₄-A12-Cy7 was once again demonstrated, and it was found that the metabolism time of the probe in the area of interest was about 120 h. The mouse organs were isolated at 6 h and 120 h after the injection of the nanoprobe. As shown in FIG. 11B, the signal was strongest in the liver at 6 h, followed by the spleen and kidney; As shown in FIG. 11C, only weak signal remained in the liver and intestine at 120 h.

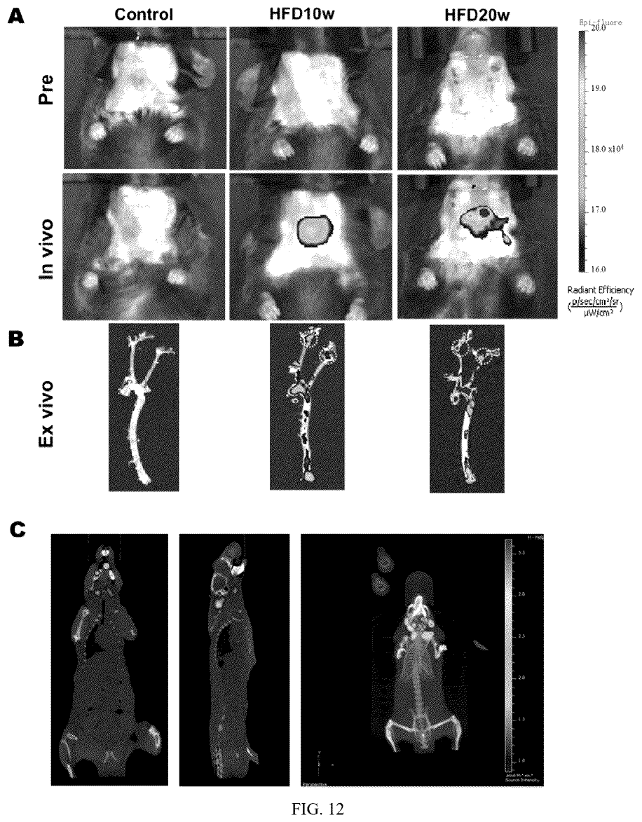

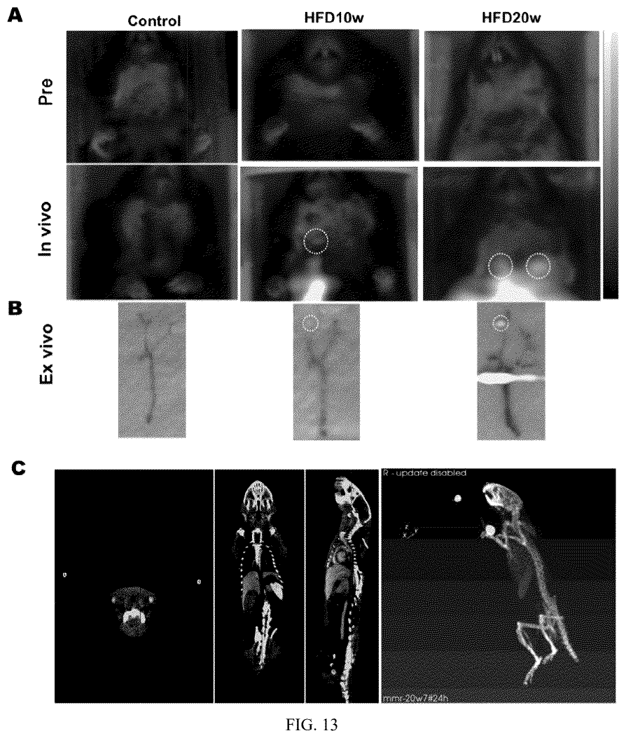

As shown in FIGS. 12 and 13, compared with the control group, the MPI and IVIS signals of carotid plaques in the atherosclerotic mice fed with high-fat diet for 10 w and 20 w were significantly higher than those before the probe injection. The MPI and IVIS signals of atherosclerotic mice fed with high-fat diet for 20 w were higher than those of model mice fed with high-fat diet for 10 w. This shows that the nanoprobe can achieve dynamic monitoring and early warning of carotid plaques through FLI/MPI/CTA multi-modal imaging.

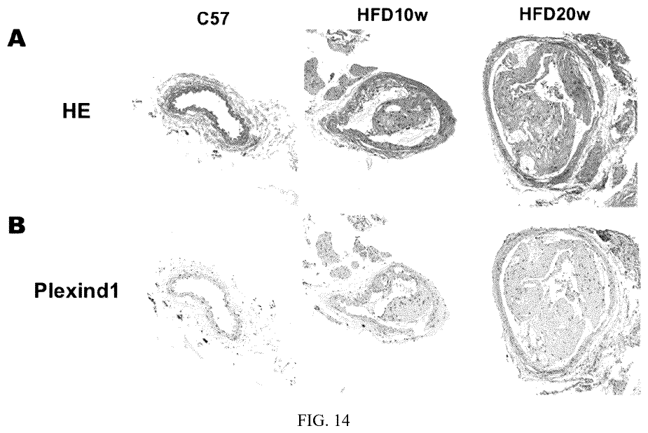

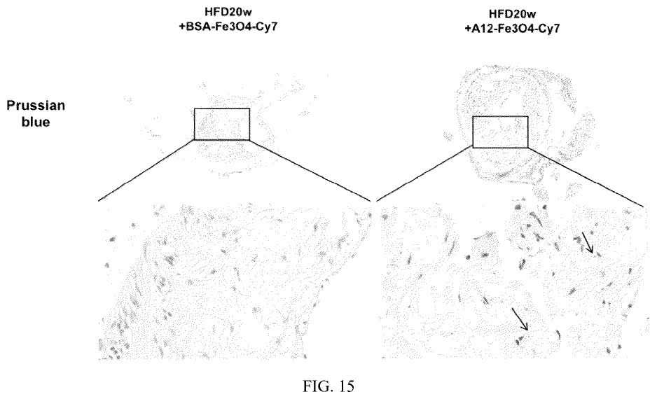

FIG. 14 shows the images of the results of HE staining and plexind1 immunohistochemical staining of the histopathological sections from carotid bifurcation sites in C57 control mice and atherosclerotic mice fed with high-fat diet for 10 w and 20 w in Example 1. It can be seen from the figure that with the prolongation of high-fat feeding time, the plaque progresses and the plexind1-positive areas increase, which is consistent with the trend of MPI and IVIS signals; FIG. 15 shows the images of Prussian blue staining results of histopathological sections of atherosclerotic plaques at carotid bifurcation sites after injection of non-targeting control probe Fe₃O₄-BSA-Cy7 and targeting nanoprobe Fe₃O₄-A12-Cy7 for 24 h in atherosclerotic mice fed with high-fat diet for 20 w in Example 1. The former was not Prussian blue positive while the latter was Prussian blue positive, indicating that Fe₃O₄-A12-Cy7 targeted the location of interest and deposited there compared with the non-targeting control probe.

A transmission electron microscope (JEM-1200EX, JEOL, Tokyo, Japan) was used to measure the nanoparticle size of the Fe₃O₄-A12-Cy7 probe in Examples 2-5. The results are shown in FIGS. 16-19, the particle size of the nanoprobe Fe₃O₄-A12-Cy7 is 5 to 40 nm.

The probe of the present invention is Fe₃O₄-A12-Cy7, wherein the A12 in the probe Fe₃O₄-A12-Cy7 is A12 protein, and the protein A12 is a single-domain antibody that can specifically bind to PlexinD1, that is, the probe Fe₃O₄-A12-Cy7 can specifically bind to PlexinD1, thus achieving the optimization of the target. The multi-modal imaging of the present invention not only has the advantages of a high sensitivity and a high spatial resolution, can realize early warning and dynamic monitoring of atherosclerotic plaques, and can also visually reflect 3D stereoscopic imaging, thereby providing theoretical and technical support for basic research.

The foregoing embodiments are only intended to illustrate rather than limit the technical solutions of the present invention. Although the present invention has been described in detail with reference to the foregoing examples, those of ordinary skill in the art should understand that: it is still possible to modify the technical solutions recorded in the foregoing embodiments, or perform equivalent substitutions for some of the technical features. However, these modifications or substitutions do not make the essence of the corresponding technical solutions deviate from the spirit and scope of the technical solutions of the embodiments of the present invention.

What is claimed is:

1. A multi-modal molecular imaging nanoprobe for early warning and dynamic monitoring of atherosclerotic plaques, characterized in that: the multi-modal molecular imaging is an in vivo multi-modal imaging method fusing MPI, FLI, and CTA; the nanoprobe is Fe₃O₄-A12-Cy7, the Fe₃O₄ is superparamagnetic iron oxide for MPI imaging; a particle size of the Fe₃O₄ is 20 nm, and the Fe₃O₄ exists in the form of Fe₃O₄@PEG·COOH; Cy7 is a fluorescent group for FLI imaging; A12 is a single-domain antibody that specifically binds to PlexinD1, and the sequence of the A12 protein is shown in SEQ ID NO:1.

2. A method for preparing the multi-modal molecular imaging nanoprobe for early warning and dynamic monitoring of atherosclerotic plaques according to claim 1, characterized in that the method comprises the following steps:
(1) pretreatment, comprising leaving a Fe₃O₄ solution to stand, then discarding a supernatant, washing with MES and performing resuspension; the particle size of Fe₃O₄ in the Fe₃O₄ solution is 20 nm, and the concentration of the Fe₃O₄ solution is 0.01 to 20 mg/ml;
(2) preparation of a magnetic nanoparticle Fe₃O₄-A12, comprising adding EDC into the Fe₃O₄ obtained in step (1) under vortexing, uniformly mixing and then adding A12, and uniformly mixing and then conducting a reaction under shaking; after the reaction is completed, taking out a reaction liquid, centrifuging, discarding a supernatant, and washing with purified water to obtain the magnetic nanoparticle Fe₃O₄-A12; the method for preparing the A12 comprises the following steps: \textcircled{1}inserting an A12 gene into an expression vector pET30a, and then transforming same to a BL21 expression strain; \textcircled{2} spreading the expression strain containing A12 plasmid onto an LB plate which is placed upside down in an incubator; \textcircled{3} selecting a single clone from the plate above, inoculating the single clone into an LB culture medium for culture, adding IPTG, and then inducing expression; \textcircled{4} centrifuging a culture liquid after induction, removing a supernatant, adding a PBS solution to resuspend a precipitate, adding a SDS-PAGE loading buffer, heating a sample, then centrifuging and taking a supernatant for electrophoresis;
(3) preparation of a nanoprobe Fe₃O₄-A12-Cy7, comprising adding Cy7-NHS to the magnetic nanoparticle Fe₃O₄-A12 obtained in step (2), reacting under shaking, centrifuging, discarding a supernatant, and washing with purified water to obtain the nanoprobe Fe₃O₄-A12-Cy7.

3. The method for preparing the multi-modal molecular imaging nanoprobe for early warning and dynamic monitoring of atherosclerotic plaques according to claim 2, characterized in that in step (2), the mass ratio of the Fe₃O₄ to the A12 is 5 to 20; the temperature of the reaction is 28-40° C., and the reaction time is 10-30 h.

4. The method for preparing the multi-modal molecular imaging nanoprobe for early warning and dynamic monitoring of atherosclerotic plaques according to claim 2, characterized in that in step (3), the mass ratio of the Fe₃O₄ to the Cy7 is 10 to 50:1.

5. The method for preparing the multi-modal molecular imaging nanoprobe for early warning and dynamic monitoring of atherosclerotic plaques according to claim 4, characterized in that in step (3), the particle size of the nanoprobe Fe₃O₄-A12-Cy7 is 20 nm; the potential of the nanoprobe Fe₃O₄-A12-Cy7 is-27 to 1 mV.

6. Use of the multi-modal molecular imaging nanoprobe for early warning and dynamic monitoring of atherosclerotic plaques according to claim 1 in the preparation of an early warning reagent or a diagnostic reagent for atherosclerotic plaques.

7. Use of the multi-modal molecular imaging nanoprobe for early warning and dynamic monitoring of atherosclerotic plaques according to claim 1 in the preparation of a therapeutic efficacy monitoring reagent for atherosclerotic plaques.
